SEQUENCE LISTING (6 sequences)

SEQ ID NO: 1 - protein, 24 aa, Artificial Sequence
  Synthetic Sequence
AEMKSLSQRSVPNTCTLVMCSPTE

SEQ ID NO: 2 - protein, 18 aa, Artificial Sequence
  Synthetic Sequence
VPVWKDAETTLFCASDAK

SEQ ID NO: 3 - protein, 18 aa, Artificial Sequence
  Synthetic Sequence
LVWEDLRNLCLFSYRLLR

SEQ ID NO: 4 - protein, 7 aa, Artificial Sequence
  Synthetic Sequence
TCTLVMC

SEQ ID NO: 5 - protein, 6 aa, Artificial Sequence
  Synthetic Sequence
LCLFSY

SEQ ID NO: 6 - protein, 6 aa, Artificial Sequence
  Synthetic Sequence
TTLFCA

(19) United States
(12) Patent Application Publication
(10) Pub. No.: US 2013/0184202 A1
(43) Pub. Date: Jul. 18, 2013

(54) Peptide Inhibitors and Methods of Use Thereof

(71) Applicants: Changjiang Guo, Cherry Hill, NJ (US); Andrew J. Gow, Princeton, NJ (US)

(72) Inventors: Changjiang Guo, Cherry Hill, NJ (US); Andrew J. Gow, Princeton, NJ (US)

(21) Appl. No.: 13/368,833

(22) Filed: Feb. 8, 2012

Related U.S. Application Data
(60) Provisional application No. 61/440,555, filed on Feb. 8, 2011.

(51) Int. Cl. C07K 14/785; A61K 38/17; A61K 45/06; C07K 7/08; A61K 38/10

(52) U.S. Cl. CPC C07K 14/785; C07K 7/08; A61K 38/10; A61K 45/06; A61K 38/395
USPC 514/3.8; 530/325; 530/326; 514/1.1; 530/409; 530/300; 514/21.4; 530/333

(57) ABSTRACT

Peptides and methods of use thereof are provided.

This application claims priority under 35 U.S.C. §119(e) to U.S. Provisional Patent Application No. 61/440,555, filed Feb. 8, 2011. The foregoing application is incorporated by reference herein.

This invention was made with government support under Grant No. HL074115 awarded by the National Institutes of Health. The government has certain rights in the invention.

### FIELD OF THE INVENTION

The present invention relates to the field of protein assembly. More specifically the invention provides peptides which inhibit or disrupt protein assembly. Methods of using such peptides for the treatment of diseases or disorders are also provided.

### BACKGROUND OF THE INVENTION

Pulmonary surfactant is a surface-active mixture of phospholipid and protein secreted by alveolar type II cells. The predominant functions of pulmonary surfactant are to reduce surface tension at the air-liquid interface, permitting alveolar stability throughout the respiratory cycle. There are four unique surfactant-associated proteins (SP) designated SP-A, SP-B, SP-C, and SP-D, which can be subdivided into two groups based on structure, function, and solubility. SP-B and SP-C are hydrophobic proteins. They are involved in adsorption of lipid at the air-liquid interface. SP-A and SP-D are hydrophilic proteins and do not contain significant intrinsic biophysical activity. SP-A and SP-D are members of a growing family of proteins that plays a role in the innate or non-antibody-mediated immunity. SP-A and SP-D are referred to as collectins (collagen-like lectins) a term that has been used to describe the family of proteins whose non-lung members include serum proteins mannose-binding lectin, bovine conglutinin, and CL-43. SP-A and SP-D play an important role in the pulmonary innate immune system modulating complex interactions that occur between pathogens and host effecter cells.

### SUMMARY OF THE INVENTION

In accordance with one aspect of the instant invention, peptides modulating protein assembly or quaternary structure are provided. In a particular embodiment, the peptide comprises an amino acid sequence having at least 90% homology, particularly 100% identity, to a region of a target protein, wherein the region comprises a hydrophobic region (e.g., at least five hydrophobic amino acids), and wherein the hydrophobic region comprises at least one cysteine. Compositions comprising the peptides of the instant invention are also provided. Methods of identifying and synthesizing the modulating peptides are also provided.

In accordance with another aspect of the instant invention, methods for inhibiting, treating, and/or preventing a disease, disorder, or infection are provided, particularly wherein the disease, disorder, or infection is associated with protein assembly or protein quaternary structure. In a particular embodiment, the method comprises administering at least one peptide of the instant invention to a subject. In yet another embodiment, the infection is an HIV infection. In still another embodiment, the disease or disorder is a pulmonary disease or disorder.

### BRIEF DESCRIPTIONS OF THE DRAWING

FIG. 1A provides a model of the structure of the SP-D monomer and a stylized representation of SP-D multimer assembly. FIG. 1B provides a hydropathy plot of SP-D.

FIG. 2A provides a Western blot of recombinant rat SP-D or mutant Ser15/20 denatured under reducing or non-reducing conditions. FIG. 2B provides a Western blot of recombinant rat SP-D or mutant Ser15/20 incubated with NEM-linked biotin.

FIG. 3A provides immunoblots of SNO-SP-D formation in broncoalveolar lavage (BAL) (top); the transnitrosation of recombinant SP-D (middle) and the transnitrosation of recombinant SP-D with increasing doses of L-SNOC or exposed to 200 μM authentic NO (bottom). FIG. 3B provides Western blots of BAL from SP-D overexpressing mice or 0.2 μM recombinant SP-D treated with L-SNOC (left); the gel filtration of the BAL samples in the left panel after SDS-PAGE (upper right); and the gel filtration of the BAL samples in the left panel after native electrophoresis (bottom right).

FIG. 4 provides an immunoblot of SNO-SP-D and total SP-D in BAL collected at days 2, 4, 7, 14, and 21 after control, saline, or bleomycin injection in Sprague-Dawley rats (FIG. 4A); an immunoblot of BAL, from Sprague-Dawley rats collected at day 4 after injection, which was subjected to electrophoresis for native gel to detect different fragments of SP-D (FIG. 4B); an immunoblot of BAL collected at day 8 after treatment from C57/BL6 mice intratracheally treated at a dose of 3 U/kg (FIG. 4C); and an immunoblot and graph of SNO-SP-D in the BAL of bleomycin-treated wild type versus bleomycin-treated iNOS^{-/-} mice (8-days post-injury).

FIG. 5A provides a graph of RAW cell chemotaxis induced by BAL from bleomycin- and saline-treated rats, optionally treated with ascorbate. The inset provides an immunoblot of the BAL SNO-SP-D content. In FIG. 5B, bleomycin and saline BAL were analyzed for chemotactic effect following pretreatment with anti-SP-D or non-immune IgG.

FIG. 6 provides a graph showing relative NF-κB activity in RAW cells after various treatments.

FIG. 7 provides a graph of mRNA induction by S-nitrosylated BAL in RAW cells after various treatments.

FIG. 8 provides a graph of the dose response of mRNA induction by S-nitrosylated BAL in RAW cells after various treatments.

FIG. 9A provides a silver-stained gel and a Western blot of BAL from SP-D overexpressing mice incubated with maltose-agarose beads in calcium replete buffer. FIG. 9B provides a graph of the mRNA induction in RAW cells following treatment with BAL, SNO-BAL, and SP-D depleted BAL.

FIG. 10 provides a graph of iNOS mRNA expression in RAW cells after treatment with OE-BAL or SNO-OE-BAL in the presence and absence of the NF-κB inhibitor caffeic acid phenethyl ester (CAPE).

FIG. 11 provides a Western blot of SP-D multimers under various treatments.

FIG. 12A provides a hydrophobicity plot of the designed peptide CJ-1 (SEQ ID NO: 1). FIG. 12B provides a Western blot of rSP-D (50 ng) incubated with or without CJ-1 (10 mM) or DTT (10 mM) at 37° C. for 20 minutes. FIG. 12C provides a Western blot showing of rSP-D (50 ng) incubated with different doses of CJ-1 (0.01-10 mM) at 37° C. for 20 minutes.

FIG. 13 provides a Western blot of 50 ng of RrSP-D with an equimolar concentration of CJ-1; CJ-2 (a scrambled version of CJ-1 with no hydrophobic pocket); or L-cysteine.

FIG. 14A provides an immunoblot of untreated RrSP-D (lane 1) or SNO-SP-D untreated (lane 2) or incubated with CJ-1 (1 μM (lane 3) or 10 μM (lane 4)). FIG. 14B provides a graph of RAW cell migration after various treatments.

FIGS. 15A and 15B provides images of a silver stained gel (FIG. 15A) and a Western blot (FIG. 15B) of gp160 LAV preincubated without (lanes 1, 3, 5) or with NEM biotin (lanes 2, 4, 6) and subjected to SDS page gel electrophoresis. FIG. 15C shows a Coomassie stained gel of HIV gp120 LAV preincubated without (lane 1) or with DTT (lane 2), L-cysteine (lane 3), CJ-3 (lane 4) CJ-4 (lane 5) or CJ-3 and CJ-4 (lane 6). FIG. 15D provides a graph showing HIV reverse transcriptase (RT) activity in the supernatants of cells infected with the HIV X4 strain NL4-3 preincubated without (control) or with peptide CJ-2, CJ-3, or CJ-4.

FIG. 16 provides a schematic of a representative hydrophobicity plot.

FIG. 17 shows mRNA induction in macrophage in the presence of CJ-1 or SNO-CJ-1 in the presence (FIG. 17B) or absence (FIG. 17A) of LPS stimulation.

### DETAILED DESCRIPTION OF THE INVENTION

SP-D plays a key role in mediating pathogen clearance and the pulmonary inflammatory response. Animals lacking SP-D develop spontaneous inflammation within the lung and consequently an emphysematous-like disease. Nitric oxide (NO), via a process termed S-nitrosylation, modifies the tail domain of SP-D. The target of NO, the cysteines residues number 15 and 20, are located in the N-terminus of the protein. It is the tail domain, or N-terminus, that controls the multimeric state of the protein via a series of intramolecular sulfhydryl bonds. The S-nitrosylation of cysteines 15 and/or 20 leads to disruption of these bonds and causes the SP-D dodecamer to disassemble into its constituent trimers. The disassembly exposes the tail domain of the protein, which is normally buried within the center of the dodecamer. In this way, S-nitrosylation makes SP-D able to bind to the CD91/calreticulin receptor complex. The S-nitrosylated form of SP-D may be referred to as SNO-SP-D. By initiating binding of SP-D to the CD91/calreticulin, SNO-SP-D switches SP-D from an inflammatory inhibitor to an activator. It is shown herein that these modifications are functionally relevant in animal models of injury and disease and their occurrence within human inflammatory disease is also demonstrated.

This model of SP-D regulation via NO-mediated modification of structurally relevant cysteine residues represents an entirely novel mechanism of the control of protein function. One of the first identified factors in controlling protein structure was the disulfide bond within insulin. It has become routine to consider which cysteines may be involved in disulfide formation when considering a primary amino acid sequence. When a disulfide bond is formed a formal two-electron oxidation step is required, catalyzed by protein disulfide isomerase. However, the only structural requirement is that the two thiols be vicinal within the folding protein. Once a disulfide bond has been formed the thiols are not readily available for reaction. The thiols of cysteine residues not involved in disulfide bonds are available for simple modifications such as alkylation. This is the basis of a number of post-translational modifications such as isoprenylation, methylation, and geranylation. Recently, it has become increasingly clear that S-nitrosylation, the addition of a nitric oxide moiety to a thiol residue, is another important post-translational modification. The work with SP-D presented herein has shown that the presence of a thiol residue within a hydrophobic pocket can serve to stabilize protein quaternary structure via a sulfhydryl hydrogen bond and that this bond can be regulated via S-nitrosylation.

The present invention provides a specific mechanism for protein assembly. The involvement of critical cysteine residues within a hydrophobic domain of a protein stabilizes its quaternary structure. These residues interact via a specialized form of hydrogen bond. Such specialized intrathiol hydrogen bonds within hydrophobic domains are a fundamental mechanism of quaternary protein structure. By designing peptides that can substitute for one of the partners within such a bond, their disruption is targeted and a specific protein multimer (homomultimeric protein or heteromultimeric protein) is destabilized. Furthermore, these sulfhydryl bonds are a target for NO modification via S-nitrosylation and through the use of designed peptides NO delivery can be targeted.

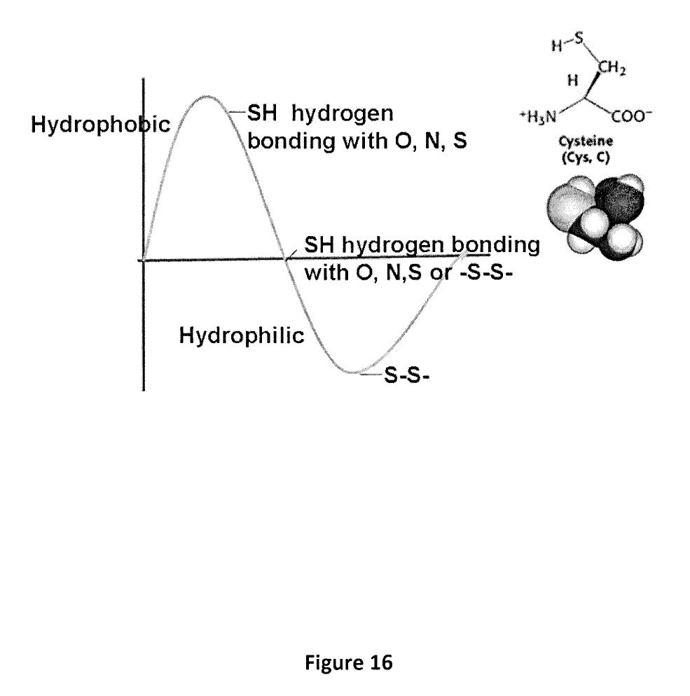

As stated hereinabove, the instant invention is based on this new understanding of mechanism of protein assembly. The peptide sequence is directly derived from the original protein to target this hydrophobic region. In a particular embodiment, the peptide is alpha-helical in which there are a hydrophobic and a hydrophilic pocket. For example, the peptide may target a hydrophobic and hydrophilic interacting region of the protein (see, e.g., FIG. 16). In a particular embodiment, the peptide comprises at least one cysteine residue in the hydrophobic area. Cysteine residue(s) will be specifically selected and these residues are buried in the hydrophobic portion. By designing peptides that mimic both the hydrophobic region and the vicinal sulfhydryl one can either disrupt multimeric assembly or mimic functional association to achieve signaling. This technology allows one to highlight a sequence through the first dimensional structure of a protein and turn it into the disruptive peptide. Indeed, the designed SP-D peptide described herein was based on this technology and is shown to disrupt SP-D structure.

Therefore, in accordance with the instant invention, methods of generating a peptide modulator of a multimeric protein are provided. In a particular embodiment, the method comprises a) identifying a hydrophobic region within a protein of the multimeric protein complex, wherein the hydrophobic region comprises at least one cysteine, and b) synthesizing a peptide (e.g., as defined hereinbelow) comprising the hydrophobic region and the cysteine residue. In a particular embodiment, the synthesized peptide has at least 90% homology with the multimeric protein.

The present invention provides a peptide having the amino acid sequence AEMKSLSQRSVPNTCTLVMCSPTE (SEQ ID NO: 1) that disrupts the innate immune regulatory protein surfactant protein-D (SP-D) structure. The peptide interacts with SP-D structure to regulate modified surfactant protein-D function. As stated hereinabove, the present invention also encompasses methods to design peptide inhibitors of other similarly assembled proteins, such as viral envelope proteins (e.g., HIV gp160), through this mechanism.

The involvement of critical cysteine residues within a hydrophobic domain of the protein stabilizes its quaternary structures. Indeed, in a particular embodiment, it is shown herein that cysteine residues located within a hydrophobic region and maintained in a reduced state are critical in maintaining the subunit assembly of multimeric proteins. These residues interact via a specialized form of hydrogen bond in the multimerization of these proteins. The positioning of cysteine residues within hydrophobic regions such that the sulfhydryl side chains are vicinal within the assembled multimer favors the formation of a sulfhydryl hydrogen bond. Such specialized intrathiol hydrogen bonds, within hydrophobic domains are a fundamental mechanism of maintaining quaternary structure formation. By designing peptides targeting these sites, multimeric protein assembly can be disrupted by substituting for these bonds. Specifically, this principle has been demonstrated herein using surfactant protein-D, a key regulator of innate immunity within the lung, as a target protein. The design methodology utilized for disrupting SP-D structure can be translated to other sulfhydryl bonded structures. For example, the peptides can be used to target and disrupt other proteins that form trimers and/or multimers such as viral proteins of HIV (e.g., gp160) and hepatitis C virus (HCV) and collagen (e.g., collagen IV). These peptides may be use clinically as a therapy and the target of the cysteine containing motif may be used as a unique drug discovery tool or a vaccine discovery tool. This technology also has utility in bronchopulmonary dysplasia therapy, and in inflammation, emphysema, asthma, and chronic infections such as HIV and HCV. The SP-D peptides of the instant invention may be used to treat or inhibit bronchopulmonary dysplasia, asthma, chronic obstructive pulmonary disease (COPD), acute respiratory distress syndrome (ARDS), and other chronic or disruptive pulmonary diseases.

### DEFINITIONS

The term “substantially pure” refers to a preparation comprising at least 50-60% by weight of a given material (e.g., nucleic acid, oligonucleotide, protein, peptide, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like).

The term “isolated protein” or “isolated peptide” refers primarily to a protein, polypeptide, or peptide produced by expression of a nucleic acid molecule, sufficiently separated from other proteins with which it would naturally be associated, chemically synthesized, or otherwise generated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in, e.g., “Remington's Pharmaceutical Sciences” (Ed. Gennaro; Mack Publishing, Easton, Pa.) and “Remington: The Science and Practice of Pharmacy” (Ed. Troy; Lippincott Williams & Wilkins, Baltimore, Md.).

As used herein, an “anti-HIV compound” is a compound which inhibits HIV. Examples of an anti-HIV compound include, without limitation, (I) nucleoside-analog reverse transcriptase inhibitors (NRTIs; e.g., AZT (zidovudine, RETROVIR®), lamivudine (3TC, EPIVIR®), emtricitabine (EMTRIVA®), dideoxycytidine (ddC, zalcitabine, HIVID®), 2′,3′-dideoxyinosine (ddI, VIDEX®), tenofovir DF (VIREAD®), stavudine (d4T, ZERIT®), abacavir (1592U89; ZIAGEN®), adefovir dipivoxil (bis(POM)-PMEA; PREVON®), lobucavir (BMS-180194), BCH-10652, emitricitabine, elvucitabine, and lodenosine (FddA; 2′-beta-fluoro-2′,3′-dideoxyadenosine)), (II) non-nucleoside reverse transcriptase inhibitors (NNRTIs; e.g., delavirdine (BHAP, U-90152; RESCRIPTOR®), efavirenz (DMP-266, SUSTIVA®), nevirapine (VIRAMUNE®), PNU-142721, capravirine (S-1153, AG-1549), emivirine (+)-calanolide A (NSC-675451) and B, etravirine (TMC-125), DAPY (TMC120), BILR-355 BS, PHI-236, and PHI-443 (TMC-278)), (III) protease inhibitors (PIs; e.g., amprenavir (141W94, AGENERASE®), tipranivir (PNU-140690, APTIVUS®), indinavir (MK-639; CRIXIVAN®), saquinavir (INVIRASE®, FORTOVASE®), fosamprenavir (LEXIVA®), lopinavir (ABT-378), ritonavir (ABT-538, NORVIR®), atazanavir (REYATAZ®), nelfinavir (AG-1343, VIRACEPT®), lasinavir (BMS-234475/CGP-61755), BMS-2322623, GW-640385X (VX-385), AG-001859, and SM-309515), and (IV) fusion inhibitors (FIs; e.g., T-20 (DP-178, FUZEON®) and T-1249). As used herein, the term “nucleoside-analog reverse transcriptase inhibitors” (NRTIs) refers to nucleosides and nucleotides and analogues thereof that inhibit the activity of HIV-1 reverse transcriptase. As used herein, NNRTIs are allosteric inhibitors which bind reversibly at a nonsubstrate-binding site on the HIV reverse transcriptase, thereby altering the shape of the active site or blocking polymerase activity. As used herein, the term “protease inhibitor” refers to inhibitors of the HIV-1 protease. As used herein, “fusion inhibitors” are compounds, such as peptides, which act by binding to HIV envelope protein and blocking the structural changes necessary for the virus to fuse with the host cell. Anti-HIV compounds also include HIV vaccines such as, without limitation, ALVAC® HIV (vCP1521), AIDSVAX®B/E (gp120), and combinations thereof. Anti-HIV compounds also include HIV antibodies (e.g., antibodies against gp120 or gp41 (e.g., VCR01 (Zhou et al. (Science (2010) 329:811-7), PG9 and PG16 (Doores et al. (J. Virol. (2010) 84:10510-21), and see also Walker et al. (Science (2009) 326:285-9), particularly broadly neutralizing antibodies.

As used herein, the term “antibiotic” refers to antimicrobial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, the terms “host,” “subject,” and “patient” refer to any animal, including humans.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

A “therapeutically effective amount” of a compound or a pharmaceutical composition refers to an amount effective to prevent, inhibit, treat, or lessen the symptoms of a particular disorder or disease.

### Peptides and Uses

The peptides of the present invention may be prepared in a variety of ways, according to known methods. The peptides may be purified from appropriate sources (e.g., bacterial or animal cultured cells or tissues, optionally transformed) by immunoaffinity purification. The availability of nucleic acid molecules encoding the peptides enables production of the protein using in vitro expression methods and cell-free expression systems known in the art. In vitro transcription and translation systems are commercially available, e.g., from Promega Biotech (Madison, Wis.) or Gibco-BRL (Gaithersburg, Md.).

Larger quantities of peptides may be produced by expression in a suitable prokaryotic or eukaryotic system. For example, part or all of a DNA molecule encoding for a peptide may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

Peptides produced by gene expression in a recombinant prokaryotic or eukaryotic system may be purified according to methods known in the art. A commercially available expression/secretion system can be used, whereby the recombinant protein is expressed and thereafter secreted from the host cell, and readily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemaglutinin epitope. Such methods are commonly used by skilled practitioners.

In a particular embodiment, the peptides of the instant invention are chemically synthesized. Such methods are commonly used by skilled practitioners. For example, the peptides may be synthesized using a solid-phase method. The chemically synthesized peptides may then be purified (e.g., by HPLC).

Peptides of the invention, prepared by the aforementioned methods, may be analyzed and verified according to standard procedures. For example, such protein may be subjected to amino acid sequence analysis, according to known methods.

The peptides of the instant invention may be from about 10 to about 100 amino acids, about 10 to about 50 amino acids, about 10 to about 30 amino acids, about 10 to about 25 amino acids, about 15 to about 30 amino acids, or about 15 to about 25 amino acids in length.

The peptides of the instant invention comprise a hydrophobic region. Within the hydrophobic region, the peptides of the instant invention comprise at least one cysteine. In a particular embodiment, the hydrophobic region is at least 5 amino acids in length, particularly about 5 to about 10 amino acids in length. Hydrophobic amino acids include very hydrophobic amino acids (Phe, Ile, Trp, Leu, Val, and Met) and less hydrophobic amino acids (Tyr, Cys, Ala, His, and Thr) (i.e., wherein Gly is considered neutral). In a particular embodiment, the hydrophobic region comprises only hydrophobic amino acids. In a particular embodiment, the hydrophobic region comprises at least 5 hydrophobic amino acids in addition to the cysteine residue(s). By way of example, the CJ-1 peptide of the instant invention comprises a hydrophobic region having the sequence TCTLVMC (SEQ ID NO: 4). Additionally, the CJ-3 peptide of the instant invention comprises a hydrophobic region having the sequence TTLFCA and the CJ-4 peptide comprises a hydrophobic region having the sequence LCLFSY (SEQ ID NO: 5). The hydrophobic region of the peptides of the instant invention may also be determined by a hydrophobicity plot, wherein the hydrophobic region is the region (e.g., at least 5 consecutive amino acids) determined to be hydrophobic (i.e., above zero). In a particular embodiment, the remainder of the peptide comprises hydrophilic, amphiphilic, and/or other hydrophobic regions. In a particular embodiment, the remainder of the peptide comprises a hydrophilic region (e.g., at least 5 hydrophilic amino acids (Ser, Gln, Arg, Lys, Asn, Glu, Pro, Asp), optionally interrupted by 1 or 2 non-hydrophilic amino acids). The hydrophilic region of the peptides of the instant invention may also be determined by a hydrophobicity plot, wherein the hydrophilic region is the region (e.g., at least 5 consecutive amino acids) determined to be hydrophilic (i.e., below zero).

The peptides of the instant invention may comprise a sequence (see lengths above; e.g., about 15 to about 25 amino acids in length) that is at least 90%, at least 95%, at least 98%, or 100% identical to a sequence in the target polypeptide/protein. In a particular embodiment, the sequence of the peptide is at least 90%, at least 95%, at least 98%, or 100% identical to a sequence in the target polypeptide/protein. The peptide of the instant invention may comprise SEQ ID NO: 1, SEQ ID NO: 2, or SEQ ID NO: 3. The amino acid sequence of peptide of the instant invention may comprise a sequence having at least 80%, 85%, 90%, 95%, 98%, or 100% homology with the above sequences (e.g., the sequence may contain additions and/or deletions (e.g., at either end) and/or substitutions). In a particular embodiment, the peptide of the instant invention may extend beyond the above sequences at the amino and/or carboxy terminus by 1, 2, 3, 4, 5, 6, 7, 8, 9, or 10 amino acids, particularly by 1, 2, 3, 4, or 5 amino acids, particularly by 1, 2, or 3 amino acids.

The peptides of the instant invention may be nitrosylated. In a particular embodiment, at least one cysteine residue of the peptide (e.g., the cysteine in the hydrophobic region) is nitrosylated.

As stated hereinabove, the peptide of the instant invention may contain substitutions for the amino acids of the provided sequence. In a particular embodiment, these substitutions may be similar to the amino acid (i.e., a conservative change) present in the provided sequence (e.g., an acidic amino acid in place of another acidic amino acid, a basic amino acid in place of a basic amino acid, a large hydrophobic amino acid in place of a large hydrophobic, a very hydrophobic amino acid in place of a very hydrophobic, etc.). The substitutions may also comprise amino acid analogs and mimetics. In a particular embodiment, the substitutions are predicted to promote helicity or helix formation.

The peptide of the instant invention may have capping, protecting and/or stabilizing moieties at the C-terminus and/or N-terminus. Such moieties are well known in the art and include, without limitation, amidation and acetylation. The peptide template may also be lipidated or glycosylated at any amino acid (i.e., a glycopeptide). In particular, these peptides may be PEGylated to improve druggability. The number of the PEG units (NH₂(CH₂CH₂O)CH₂CH₂CO) may vary, for example, from 1 to about 50.

The peptide of the instant invention may also comprise at least one D-amino acid instead of the native L-amino acid. The peptide may comprise only D-amino acids.

The present invention also encompasses nucleic acids encoding the peptides and pharmaceutical compositions comprising at least one peptide of the instant invention and at least one pharmaceutically acceptable carrier.

The present invention also encompasses methods for preventing, inhibiting, and/or treating a disease (e.g., a pulmonary disease), disorder, or infection in a subject. The pharmaceutical compositions of the instant invention can be administered to an animal, in particular a mammal, more particularly a human, in order to treat/inhibit/prevent a viral infection (e.g., the composition may be administered before, during, or after a viral infection). The pharmaceutical compositions of the instant invention may also comprise at least one other therapeutic agent for the preventing, inhibiting, and/or treating the disease, disorder, or infection. For example, the composition may further comprise at least one antimicrobial (e.g., antibiotic) or antiviral agent (e.g., an anti-HIV agent). The additional therapeutic agent (e.g., antimicrobial agent) may also be administered in a separate composition from the peptides of the instant invention. The compositions may be administered at the same time or at different times (e.g., sequentially).

The compositions of the instant invention may be administered, in a therapeutically effective amount, to a patient in need thereof. The pharmaceutical compositions of the present invention can be administered by any suitable route, for example, by injection (e.g., parenteral, intramuscular, intravenous, or intraperitoneal administration), by oral, pulmonary (e.g., intratraechially), nasal, topical, or other modes of administration such as controlled release devices. In general, pharmaceutical compositions and carriers of the present invention comprise, among other things, pharmaceutically acceptable diluents, preservatives, stabilizing agents, solubilizers, emulsifiers, adjuvants and/or carriers. Such compositions can include diluents of various buffer content (e.g., saline, Tris HCl, acetate, phosphate), pH and ionic strength; and additives such as detergents and solubilizing agents (e.g., Tween™ 80, Polysorbate 80), anti oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. Exemplary pharmaceutical compositions and carriers are provided, e.g., in “Remington's Pharmaceutical Sciences” by E.W. Martin (Mack Pub. Co., Easton, Pa.) and “Remington: The Science And Practice Of Pharmacy” by Alfonso R. Gennaro (Lippincott Williams & Wilkins) which are herein incorporated by reference. The pharmaceutical composition of the present invention can be prepared, for example, in liquid form, aerosolized form, or can be in pill or dried powder form (e.g., lyophilized).

In yet another embodiment, the pharmaceutical compositions of the present invention can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71:105).

The present invention also encompasses methods for preventing, inhibiting, and/or treating a disease, disorder, or infection by administering at least one composition of the instant invention to a subject. In a particular embodiment, the present invention encompasses methods for preventing, inhibiting, and/or treating microbial infections (e.g., viral or bacterial), particularly HIV infections (e.g., HIV-1, HIV-2, etc.), rotaviral, HBV, and HCV infections. The pharmaceutical compositions of the instant invention can be administered to an animal, in particular a mammal, more particularly a human, in order to treat/inhibit/prevent an infection (e.g., the composition may be administered before, during, or after an infection). In a particular embodiment, the present invention encompasses methods for preventing, inhibiting, and/or treating an HIV infection in a patient comprising administration of at least one composition comprising at least one peptide of the instant invention, particularly SEQ ID NO: 2, SEQ ID NO: 3, or derivatives thereof. The pharmaceutical compositions of the instant invention may also comprise at least one other anti-microbial agent, particularly at least one other anti-HIV compound/agent. The additional anti-HIV compound may also be administered in separate composition from the anti-HIV peptides of the instant invention. The compositions may be administered at the same time or at different times (e.g., sequentially).

In a particular embodiment, the present invention encompasses methods for preventing, inhibiting, and/or treating pulmonary disorders or diseases. In a particular embodiment, the pulmonary disease or disorder is pneumonia (e.g., Pneumocystis pneumonia (PCP)), emphysema, asthma, bronchopulmonary dysplasia, chronic obstructive pulmonary disease (COPD), acute respiratory distress syndrome (ARDS), and other chronic or disruptive pulmonary diseases. In a particular embodiment, the peptide comprises SEQ ID NO: 1 or a derivative thereof. The pharmaceutical compositions of the instant invention may also comprise at least one other anti-microbial agent (e.g., an antibiotic). The additional anti-microbial compound may also be administered in separate composition from the peptides of the instant invention. The compositions may be administered at the same time or at different times (e.g., sequentially).

The dosage ranges for the administration of the compositions of the invention are those large enough to produce the desired effect (e.g., curing, relieving, treating, and/or preventing the disease, disorder, or infection and/or the symptoms associated therewith). The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The following examples describe illustrative methods of practicing the instant invention and are not intended to limit the scope of the invention in any way.

### Example 1

Guo et al. (PLoS Biology (2008) 6:e266) provides more detailed descriptions of the materials and methods for certain of the experiments described hereinbelow.

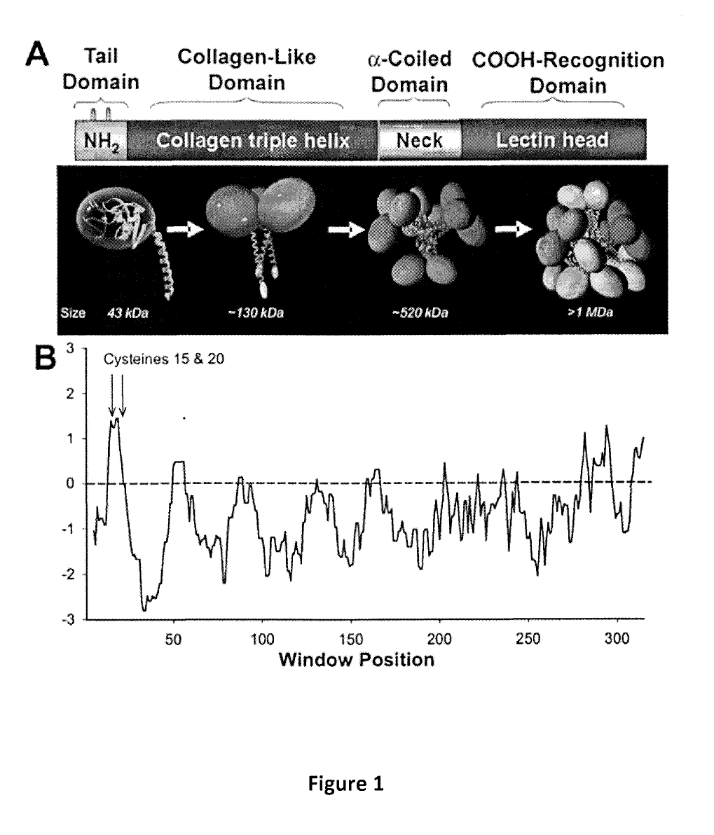

FIG. 1A provides a model of SP-D structure. The SP-D monomer (43 kDa) consists of a carbohydrate recognition domain which forms the globular head structure. This domain is connected to the collagen-like helical tail domain by a short, 30-amino acid, neck domain. At the end of the tail domain is the amino terminus in which cysteines 15 and 20 are positioned (shown as projections). FIG. 1A also provides a stylized representation of SP-D multimer assembly (note tail domains are shown shortened for ease of visualization). The head and neck domains drive the aggregation of the SP-D monomer to form a trimer of ˜130 kDa. These trimers associate to form a dodecamer (˜520 kDa). The forces holding this dodecamer together are not completely known, although there is a dependency upon the amino-terminal cysteines as mutants lacking these cysteines do not form dodecamers. These dodecamers can assemble to a multimer of greater than 1 MDa. It should be noted that neither the trimer nor the dodecamer are globular proteins, due to the presence of the long collagen tail and thus under native conditions will behave as molecules with greater molecular radius.

FIG. 1B provides a hydropathy plot of SP-D. A Kyle Doolittle hydropathy plot for the SP-D sequence was constructed using a window size of nine residues. A positive value indicates a region of hydrophobicity. The position of cysteines 15 and 20 are marked within the tail domain. As evidenced by FIG. 1B, this is the most hydrophobic portion of the molecule.

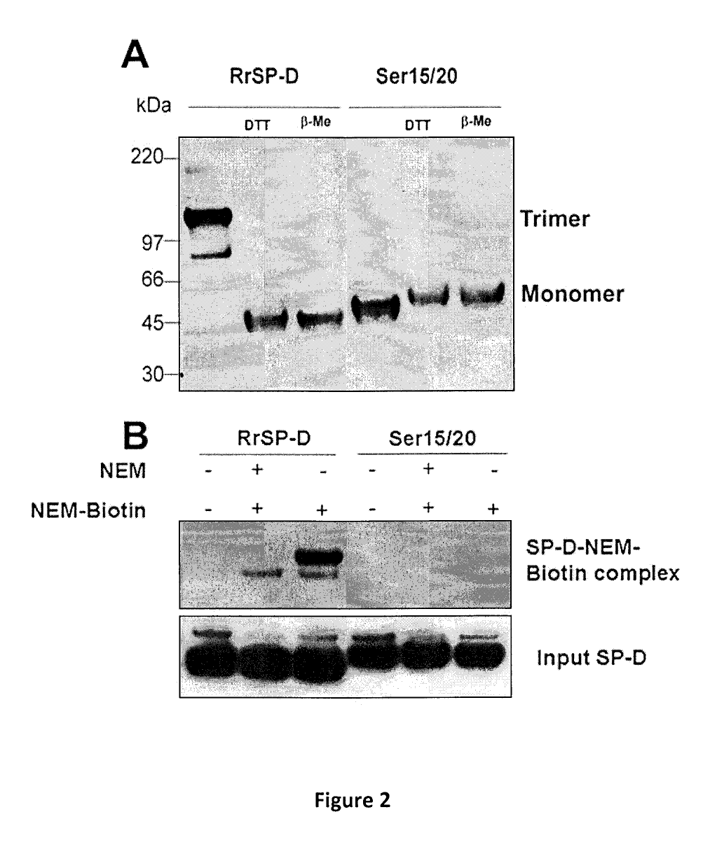

FIG. 2 demonstrates that cysteine residues 15 and 20 are critical in trimer Formation and are in a reduced state. Recombinant rat SP-D (RrSP-D) or a mutant in which cysteines 15 and 20 have been mutated to serine (Ser15/20) were denatured under reducing (using mercaptoehtanol or dithiothreitol as the reductant) and non-reducing conditions. The resultant proteins were analyzed by SDS-PAGE and Western blotting with SP-D antibody (FIG. 2A). In FIG. 2B, RrSP-D and Ser15/20 were pre-incubated with the alkylating agent N-ethyl maleimide (NEM)-linked to biotin at 37° C. for half an hour either with or without prior incubation with unlinked NEM. Biotin-labeling was determined by Western blotting following SDS-PAGE with anti-biotin antibody. These data indicate that within the recombinant protein, these two cysteines exist at least partially within a reduced state, but that the other cysteines located in the head domain are oxidized.

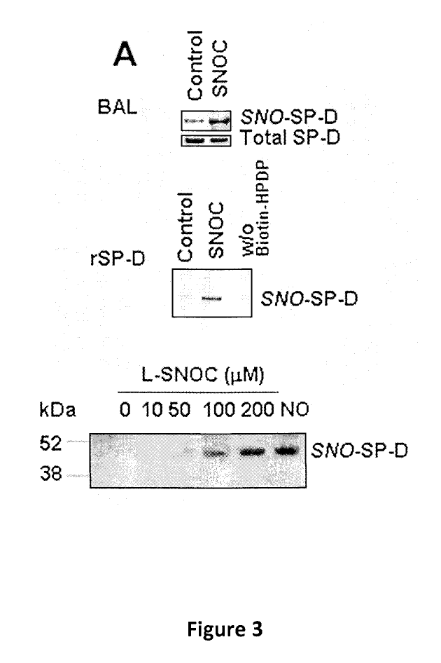

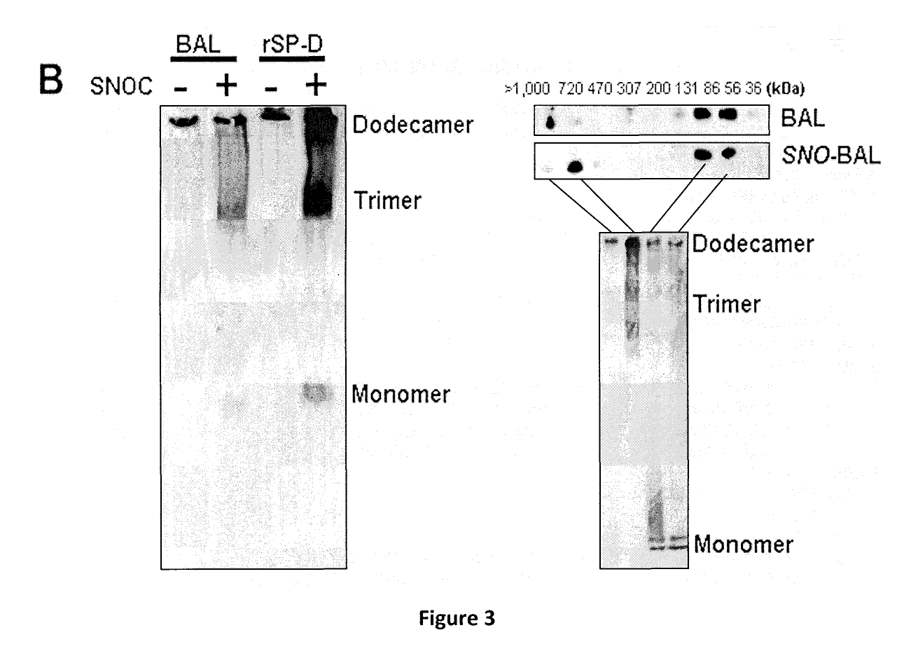

FIG. 3 demonstrates SNO-SP-D (the S-nitrosylated form of SP-D) formation in vitro and its effect on multimerization. FIG. 3A shows SNO-SP-D formation in broncoalveolar lavage (BAL). BAL from normal rats either with or without treatment with L-S-nitrocysteine (L-SNOC; 200 μM) was analyzed for SNO-SP-D content by biotin-switch assay (Jaffrey et al. (2001) Nat. Cell Biol., 3:197-197). Total SP-D content was also measured by immunoblot. FIG. 3A also shows the transnitrosation of recombinant SP-D. Control and SNOC treated recombinant SP-D (0.2 μM) were analyzed by biotin-switch assay. “W/o biotin-HPDP” represents the assay performed in the absence of biotin linked [N-(6-biotinamido)hexyl-1□-(2′ pyridyldithio)propionamide]. Recombinant SP-D (0.2 μM) was also transnitrosated with increasing doses of L-SNOC or exposed to 200 μM authentic NO and then analyzed by biotin-switch assay (FIG. 3A).

FIG. 3B shows that SNOC treatment alters the conformational state of SP-D. BAL from SP-D overexpressing mice or 0.2 μM recombinant SP-D were treated with L-SNOC and subjected to native electrophoresis and Western blot for SP-D, revealing disruption of the native multimers to dodecamers and trimers. The BAL samples in the left panel of FIG. 3B were subjected to gel-filtration. Total protein from BAL (0.75 mg) in a volume of 250 μl was resolved onto a Superdex 200 HR 10/30 column (GE Healthcare Bio-Sciences) for size-exclusion chromatography (SEC) analysis. Protein extracts were resolved at flow rate of 0.3 ml/min in 25 mM HEPES, PH 7.25, and 150 mM NaCl using an Agilent 1100 Series HLC system. Fractions (0.5 ml) were collected and concentrated with 5000 NMWL Ultrafree-MC filters (Millipore). The gel filtration column was calibrated using the following mixture of globular proteins standards: thyroglobulin (669 kDa), ferritin (440 kDa), catalase (232 kDa), aldlase (158 kDa), albumin (67 kDa), ovalbumin (43 kDa), chymotrypsin (25 kDa), and ribonuclease A (13.7 kDa). The void volume was determined from the elution migration of blue dextran (2,000 kDa). The fractions were analyzed by SDS-PAGE and Western blot for total SP-D content. The large multimers seen in control BAL are reduced in size upon SNOC treatment. Gel filtration samples containing SP-D were analyzed by native electrophoresis and Western blot, revealing that the 720-kDa fraction that arises upon SNOC treatment contains dodecamers and trimers of SP-D.

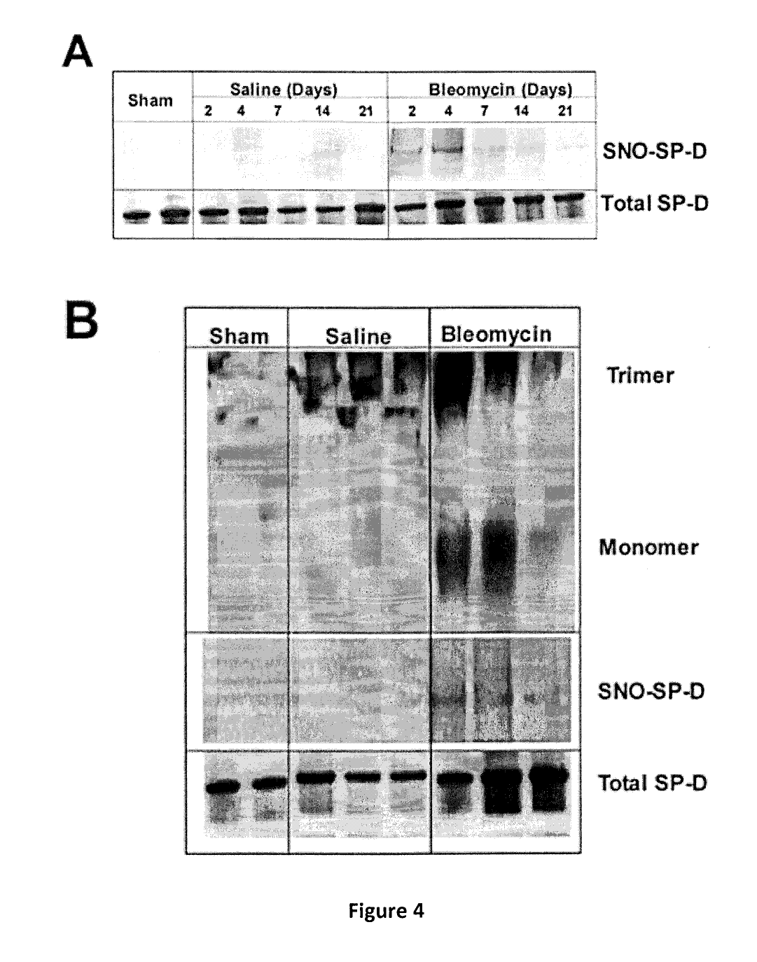

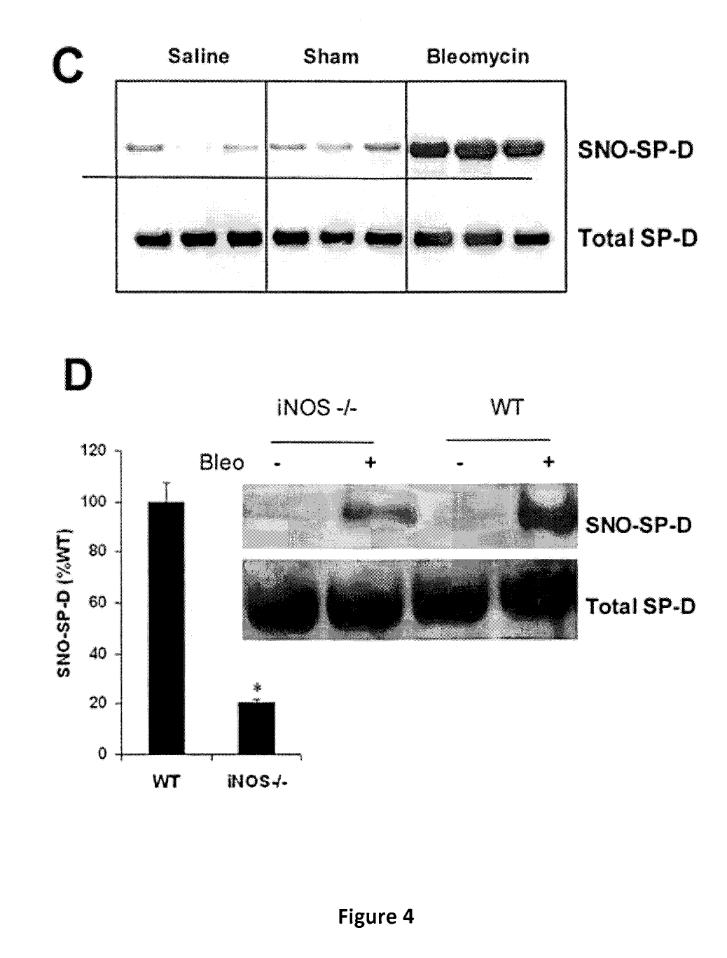

FIG. 4 shows that acute lung injury in the rodent results in SNO-SP-D Formation and multimer disruption. In FIG. 4A, untreated (control), saline, or bleomycin was intratracheally administered to Sprague-Dawley rats. BAL was collected at days 2, 4, 7, 14, and 21 after injection. BAL (upper) was subjected to biotin-switch assay to detect SNO-SP-D. Total SP-D (lower) in BAL was identified by immunoblot. In FIG. 4B, untreated (control), saline, or bleomycin was intratracheally injected at a dose of 8 U/kg in Sprague-Dawley rats. BAL was collected at day 4 after injection. BAL (upper) was subjected to electrophoresis for native gel to detect different fragments of SP-D. BAL (middle) was subjected to biotin-switch assay to detect SNO-SP-D. Total SP-D (lower) in BAL was identified by immunoblot. In FIG. 4C, untreated (control), saline, or bleomycin was administered to C57/BL6 mice intratracheally at a dose of 3 U/kg. BAL was collected at day 8 after treatment; total SNO content within the BAL was 0.2±0.22 μM in control mice and 2.1±0.88 μM in bleomycin treated. SNO-SP-D content was assessed by the biotin switch method. In FIG. 4D, SNO-SP-D in the BAL of bleomycin-treated wild type versus bleomycin-treated iNOS−/− mice (8-d post-injury) was measured by biotin-switch assay; Total input of SP-D was same between groups (asterisk represents significantly different from wild type; p<0.05). Representative blots show SNO-SP-D and total SP-D. Data are mean±SEM.

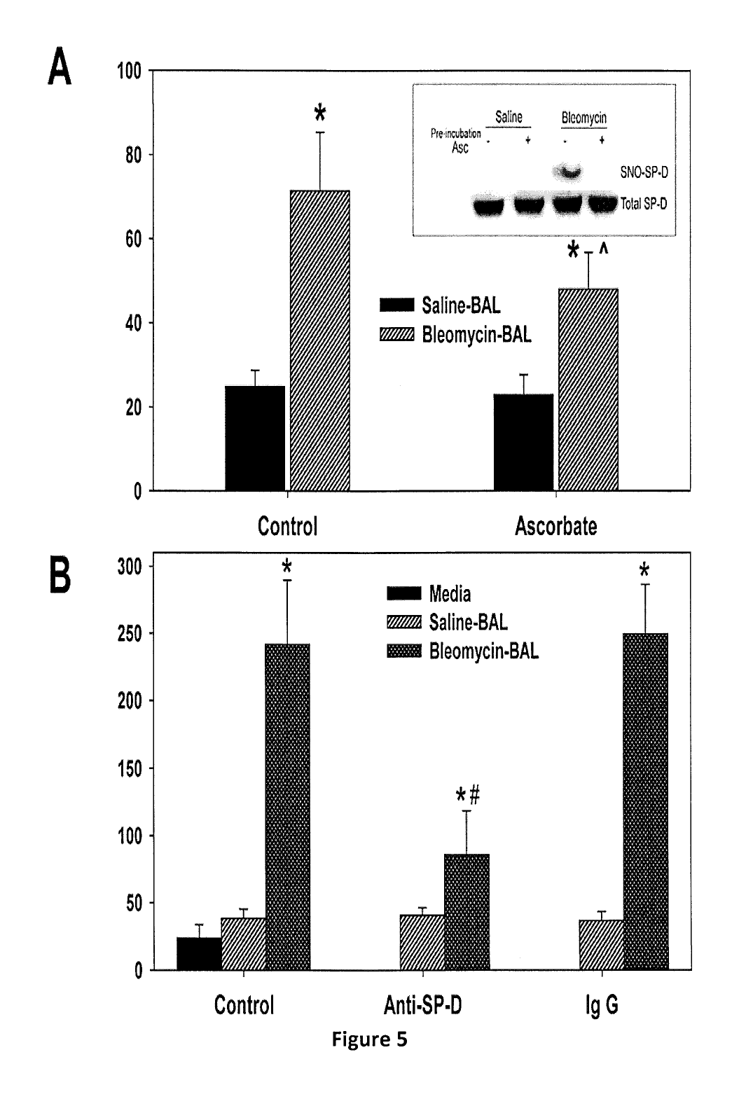

FIG. 5 shows that BAL from bleomycin-treated rats induces macrophage chemotaxis in part through SNO-SP-D. BAL from bleomycin- and saline-treated rats was analyzed for their ability to induce RAW cell chemotaxis (FIG. 5A). Bleomycin BAL induced chemotaxis to a greater extent than saline BAL; however, pretreatment with ascorbate to remove SNO abrogated this response. The effect of ascorbate treatment on BAL SNO-SP-D content is shown in the inset where samples were analyzed by biotin-switch. The importance of SP-D in this SNO-mediated increase is demonstrated by effect of SP-D immunoprecipitation (FIG. 5B). Bleomycin and saline BAL were analyzed for chemotactic effect following pretreatment with anti-SP-D or non-immune IgG. Only anti-SP-D pre-treatment reduced the increase in chemotaxis induced following bleomycin administration. (* represents significantly different from saline-BAL; # represents significantly different from control or IgG; p<0.05).

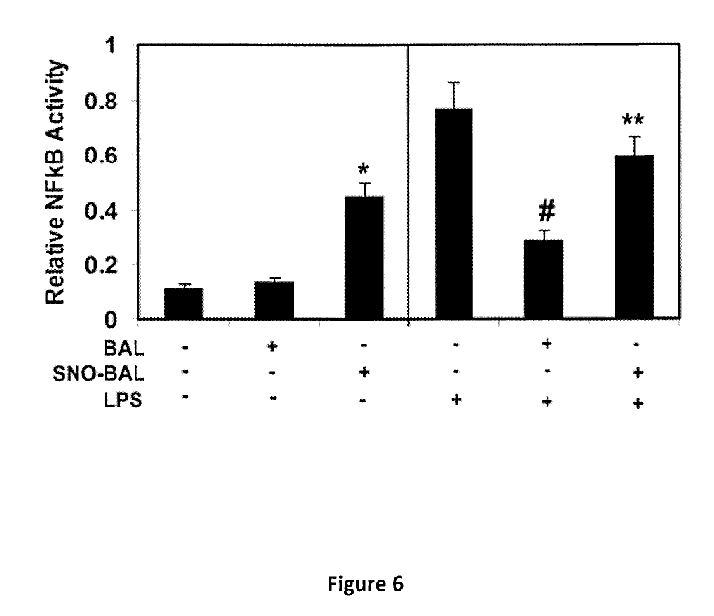

FIG. 6 shows that S-nitrosylation of BAL alters its effect on NF-κB activity in RAW cells. NF-κB activity was assayed in nuclear extracts from RAW cells by TransAM™ ELISA following treatment. BAL was added, either without prior transnitrosation with L-SNOC, in the presence or absence of LPS. * represents significantly different from no treatment; # significantly different from LPS alone; and ** significantly different from both LPS and LPS+BAL; (p<0.05).

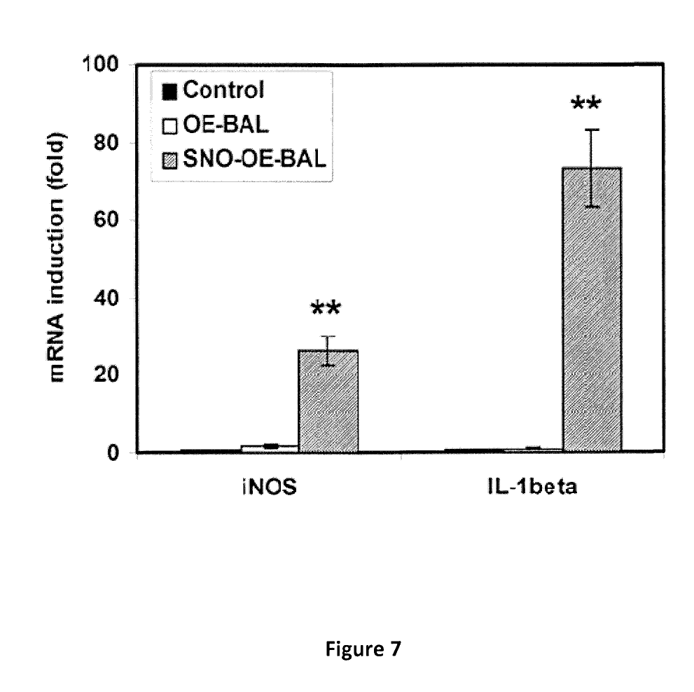

FIG. 7 shows that S-nitrosylated BAL induces iNOS and IL-1β expression in RAW cells. RAW cells were treated with BAL prepared from overexpressing mice, either with or without prior transnitrosation with L-SNOC. Following treatment for 3 hours cells were lysed and mRNA expression was measured by RT-PCR. ** represents significantly different from control, p<0.05.

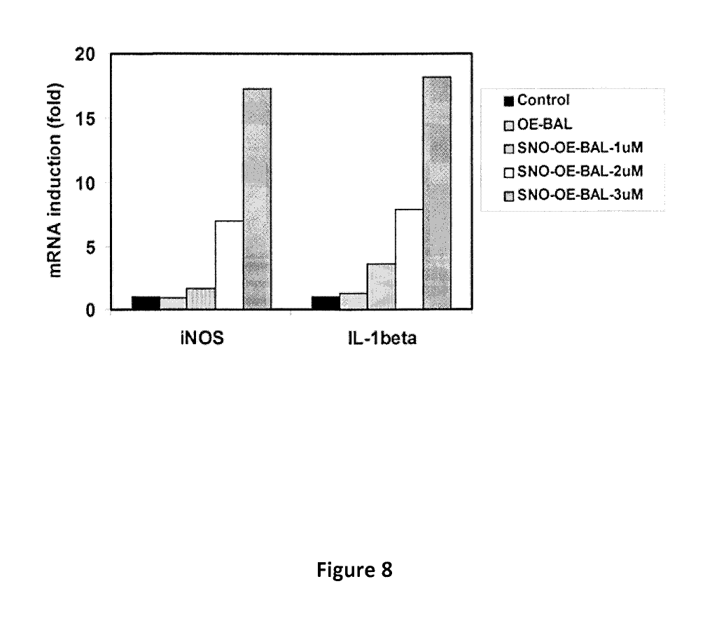

FIG. 8 shows the dose response of iNOS and IL-1β expression to SNO-BAL. RAW cells were treated with increasing doses of SNO-BAL for 3 hours. Cells were lysed and mRNA expression was measured via RT-PCR. Fold induction is calculated relative to control.

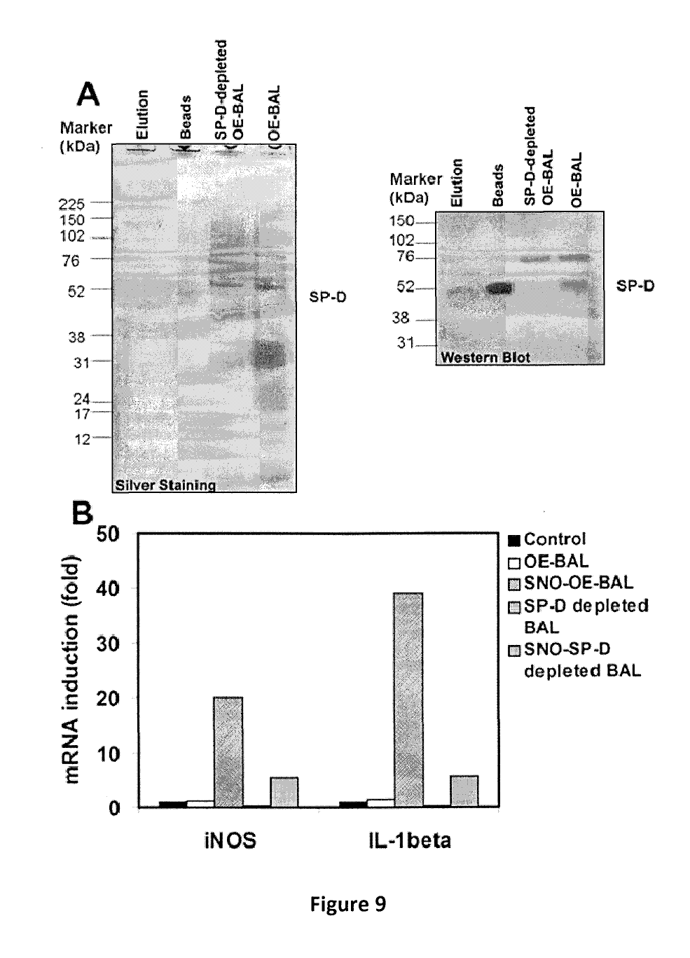

FIG. 9 shows that SNO-mediated induction of gene expression is dependent upon SP-D. SP-D as a collectin binds carbohydrates in a calcium dependent manner (FIG. 9A. BAL from SP-D overexpressing mice was incubated with maltose-agarose beads in calcium replete buffer. Following two washing steps beads were removed from the BAL by centrifugation (SP-D depleted BAL). SP-D was eluted from the beads by washing in calcium free manganese replete buffer (Elution). The efficiency of the elution was assessed by analyzing proteins that remained bound to the beads (Beads). Silver staining shows that a protein of the same molecular weight predominates within both the elution and on the beads, while few other proteins were from the BAL. Western blot demonstrates that the eluted protein is indeed SP-D and that the depleted BAL does not contain significant detectable SP-D. iNOS and IL-1β expression in RAW cells following treatment with BAL, SNO-BAL, and SP-D depleted BAL is shown in FIG. 9B. S-Nitrosylated SP-D depleted BAL had minimal effect on gene expression.

Table 1 provides a gene array analysis of SNO-overexpressing-BAL (SNO-OE-BAL) regulated genes in RAW264.7 macrophages. RAW264.7 macrophages were incubated with or without OE-BAL (100 ug/mL) or SNO-OE-BAL (100 ug/mL) for 20 minutes following treatment with or without LPS (1 ng/mL) for additional 3 hours. Total RNA was harvested and subjected to the gene array using RT2 Profiler™ PCR Array System (SuperArray Bioscience Corporation; Frederick, Md.). Data analysis was performed using Data-Analysis-Template from Superarray Bioscience Corporation. The effect of S-nitorsylation is demonstrated by comparing the fold change in expression between the relevant OE-BAL and SNO-OE-BAL treated cells (i.e., SNO-OE-BAL expression is compared to OE-BAL expression; and SNO-OE-BAL+LPS expression is compared to OE-BAL+LPS). Fold changes of less than 2 are ignored.

TABLE 1
Gene array analysis of SNO-OE-
BAL regulated genes in RAW cells.

| | | |
|---|---|---|
| | Gene | |
| Gene Name | Symbol | Fold |

Gene expression In SNO-OE-BAL treated Raw264.7 macrophages

| | | |
|---|---|---|
| Myelocytomatosis oncogene | Myc | 17.6 |
| Nitric oxide synthase 2 | NOS2 | 16.6 |
| Interleukin-1α | IL-1α | 5.1 |
| Prostaglandin-endoperoxide synthase 2 | Ptgs2 | 3.0 |
| Vascular cell adhesion molecule 1 | Vcam1 | −7.2 |
| Fas ligand | FasL | −6.4 |
| Wingless-related MMTV integration site 2 | Wnt2 | −4.7 |
| Chemokine (C-C motif) ligand 20 | CCL20 | −3.5 |
| Chemokine (C-X-C motif) ligand 9 | CXCL9 | −3.0 |
| Fas (TNF receptor superfamily member) | Fas | −2.9 |
| Bone morphogenetic protein 4 | Bmp4 | −2.6 |
| Matrix metallopeptidase 7 | Mmp7 | −2.5 |
| Selectin, Endothelial cell | Sele | −2.2 |
| Selectin, Platelet | Selp | −2.2 |
| Bone morphogenetic protein 2 | Bmp2 | −2.1 |
| WNT 1 inducible signaling pathway protein1 | Wisp1 | −2.1 |

Gene expression in SNO-OE-BAL treated Raw264.7 macrophages in
presence of LPS (1 ng/mL)

| | | |
|---|---|---|
| Interleukin-1α | IL-1α | 171.1 |
| Myelocytomatosis oncogene | Myc | 87.3 |
| Nitric oxide synthase 2 | NOS2 | 78.3 |
| Prostaglandin-endoperoxide synthase 2 | Ptgs2 | 28.0 |
| Chemokine (C-C motif) ligand 2 | CCL2 | 17.8 |
| Fas (TNF receptor superfamily member) | Fas | 9.1 |
| Growth arrest and DNA-damage-inducible | Gadd45a | 3.7 |
| 45 alpha | | |
| Interferon regulatory factor 1 | Irf1 | 2.0 |
| Interleukin-2 receptor, alpha chain | IL-2RA | −13.9 |
| Vascular cell adhesion molecule 1 | Vcam1 | −12.9 |
| Hedgehog-interacting protein | Hhip | −7.8 |
| Chemokine (C-C motif) ligand 20 | CCl20 | −6.9 |
| Matrix metallopeptidase 10 | Mmp10 | −6.2 |
| Fibroblast growth factor 4 | Fgf4 | −5.5 |
| Chemokine (C-x-C motif) ligand 1 | CXCL1 | −5.2 |
| Wingless-related MMTV integration site 2 | Wnt2 | −5.1 |
| WNT 1 inducible signaling pathway protein1 | Wisp1 | −4.8 |
| Matrix metallopeptidase 7 | Mmp7 | −4.5 |
| Bone morphogenetic protein 4 | bmp4 | −4.4 |
| Fas ligand | FasL | −3.6 |
| Interleukin-4 receptor, alpha | IL-4RA | −3.3 |
| Cytochrome P450, family 19, subfamily a, | Cyp19a1 | −3.1 |
| polypeptide 1 | | |
| Retinal binding protein 1, cellular | Rbp1 | −3.0 |
| Selectin, Endothelial cell | Sele | −2.9 |
| Wingless-related MMTV integration site 1 | Wnt1 | −2.9 |
| Cyclin-dependent kinase inhibitor 2a | Cdkn2a | −2.5 |
| Colony stimulating factor 2 | Csf2 | −2.5 |
| Interleukin-2 | IL-2 | −2.4 |
| Baculoviral IAP repeat-containing 1a | Birc 1a | −2.2 |
| Insulin-like growth factor binding protein 3 | Igfbp3 | −2.2 |

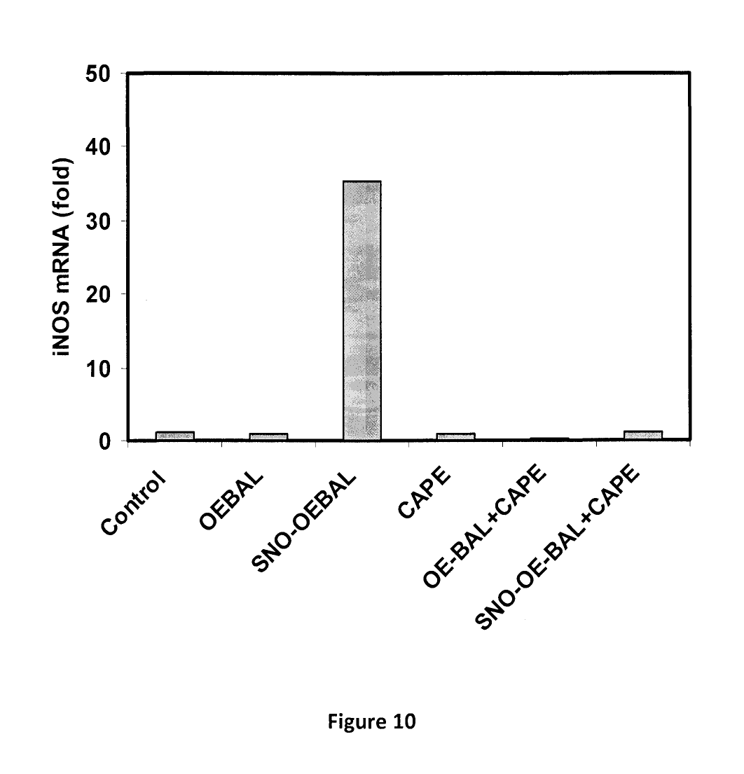

FIG. 10 shows that SNO-BAL increases iNOS expression in RAW cells via NF-κB activation. RAW cells were treated with OE-BAL or SNO-OE-BAL in the presence and absence of the NF-κB inhibitor caffeic acid phenethyl ester (CAPE). CAPE treatment completely abrogated the increase in iNOS expression mediated by SNO-OE-BAL as assessed by RT-PCR.

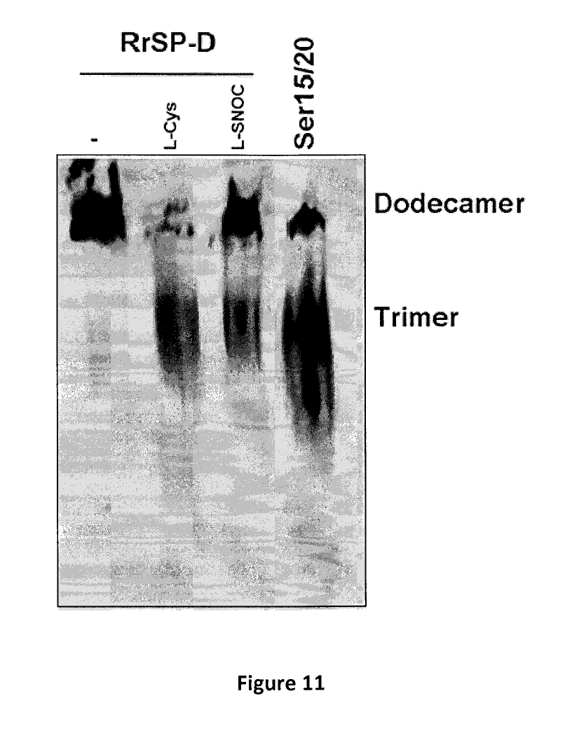

FIG. 11 shows that L-cysteine disrupts the SP-D multimer. RrSP-D was incubated with L-cysteine (25 mM) or L-S-nitroso-cysteine (25 mM) at 37° C. for 20 minutes. The resulted protein was subjected to native gel electrophoresis and western blotted for SP-D. L-cysteine, as well as L-SNOC, is capable of disrupting the SP-D multimer at high concentration, indicating that SP-D multimers are in part maintained by sulfhydryl hydrogen bonds.

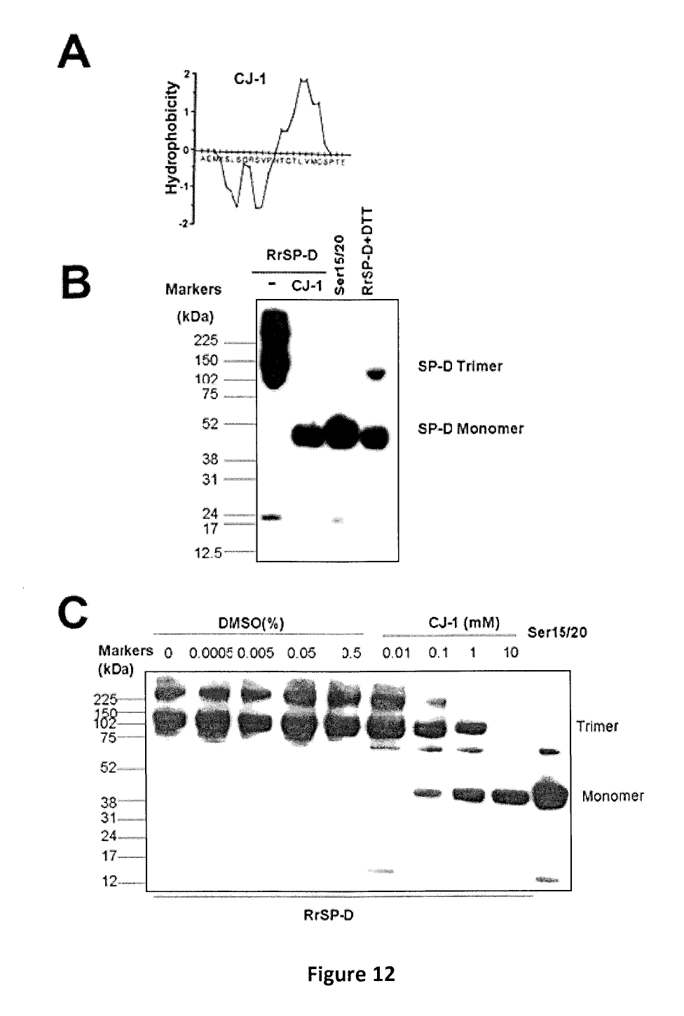

FIG. 12 shows that the designed peptide CJ-1 disrupts SP-D trimers. FIG. 12A provides a hydrophobicity plot of the designed peptide CJ-1 (SEQ ID NO: 1). CJ-1 was directly derived from SP-D sequence within which cysteine residues are located in the hydrophobic pocket. RrSP-D (50 ng) was incubated with or without CJ-1 (10 mM) or DTT (10 mM) at 37° C. for 20 minutes (FIG. 12B). The resultant protein was analyzed by SDS PAGE and Western blot for SP-D. The data demonstrated that CJ-1 disrupts SP-D trimers. FIG. 12C shows that CJ-1 disrupts SP-D in a dose-dependent manner. RrSP-D (50 ng) was incubated with different doses of CJ-1 (0.01-10 mM) at 37° C. for 20 minutes.

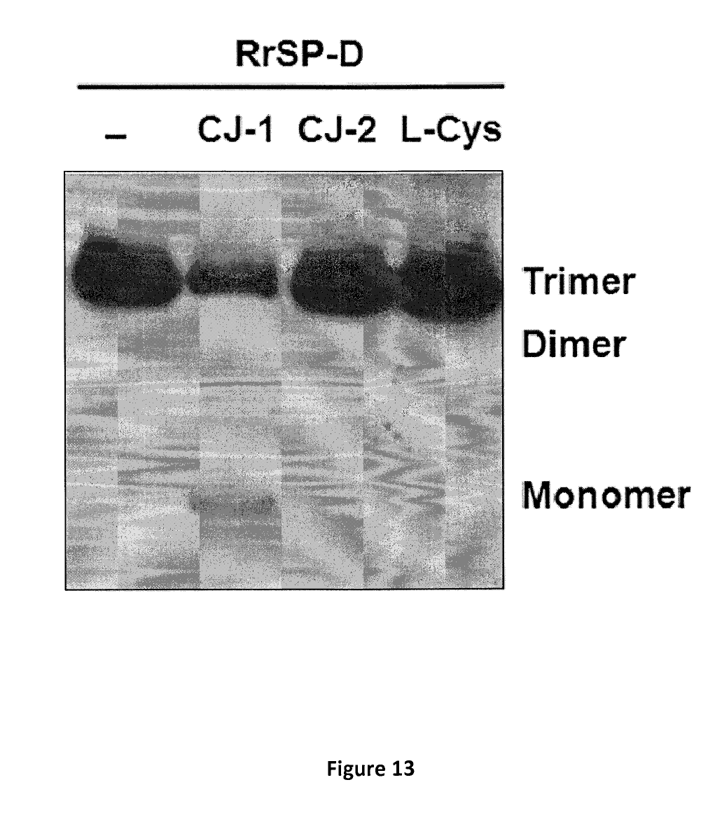

FIG. 13 shows that CJ-1 specifically disrupts SP-D multimeric state. By matching the reduced cysteines within the peptide to those in the target protein one can specifically disrupt structure. Using 50 ng of RrSP-D the effect of an equimolar concentration of CJ-1, targeted peptide; CJ-2, a scrambled version of CJ-1 with no hydrophobic pocket; or L-cysteine was analyzed. Following incubation SP-D structure was assessed by non-reducing denaturing SDS-PAGE. Only CJ-1 is capable of disrupting SP-D structure.

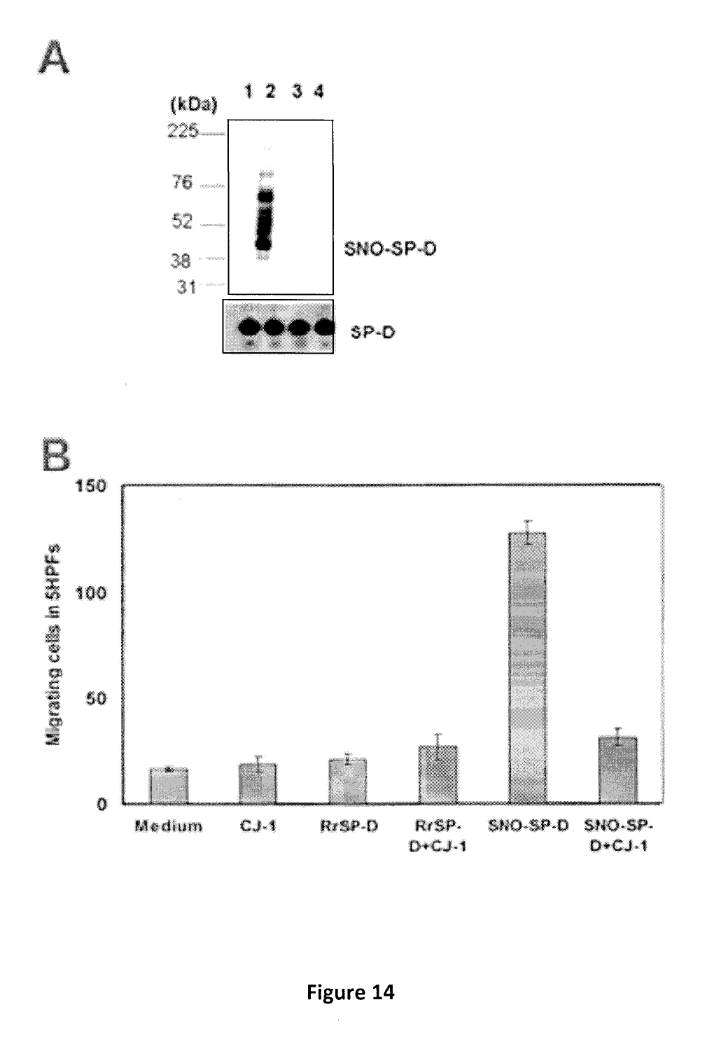

FIG. 14 shows that the designed peptide CJ-1 inhibits SNO-SP-D mediated macrophage migration. RrSP-D was incubated with 100 μM of nitric oxide for 30 minutes to generate SNO-SP-D (FIG. 14A). The resultant protein was incubated with CJ-1 (1 μM or 10 μM). The protein samples were subjected to Biotin-Switch analysis to determine SNO-SP-D content. Lane 1 untreated RrSP-D; Lane 2 NO-treated RrSP-D; Lane 3 NO-treated RrSP-D+1 μM CJ-1; Lane 4 NO-treated RrSP-D+10 μM CJ-1. The data shows that CJ-1 depleted S-nitrosothiol from SNO-SP-D. The chemotactic activity was assayed for the protein samples from FIG. 14A (FIG. 14B). The chemoattractive activity to RAW 264.7 cells was assayed using a Boydin Chamber. The data show that CJ-1 treatment inhibits the ability of SNO-SP-D to induce RAW 264.7 cell migration.

### Example 2

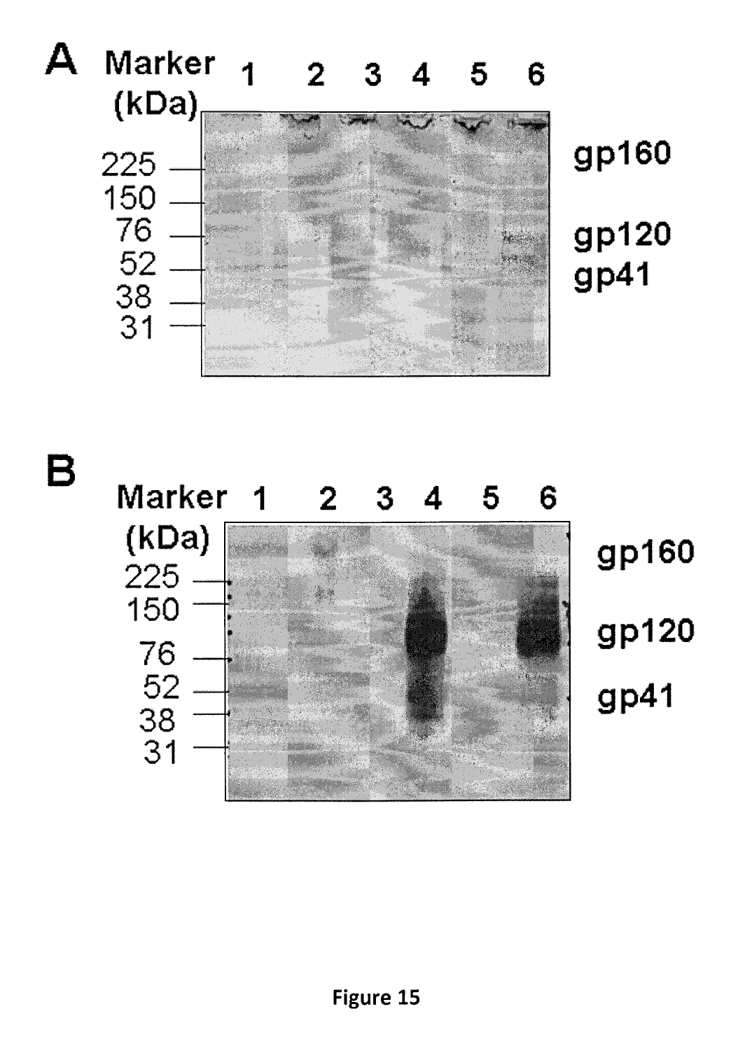

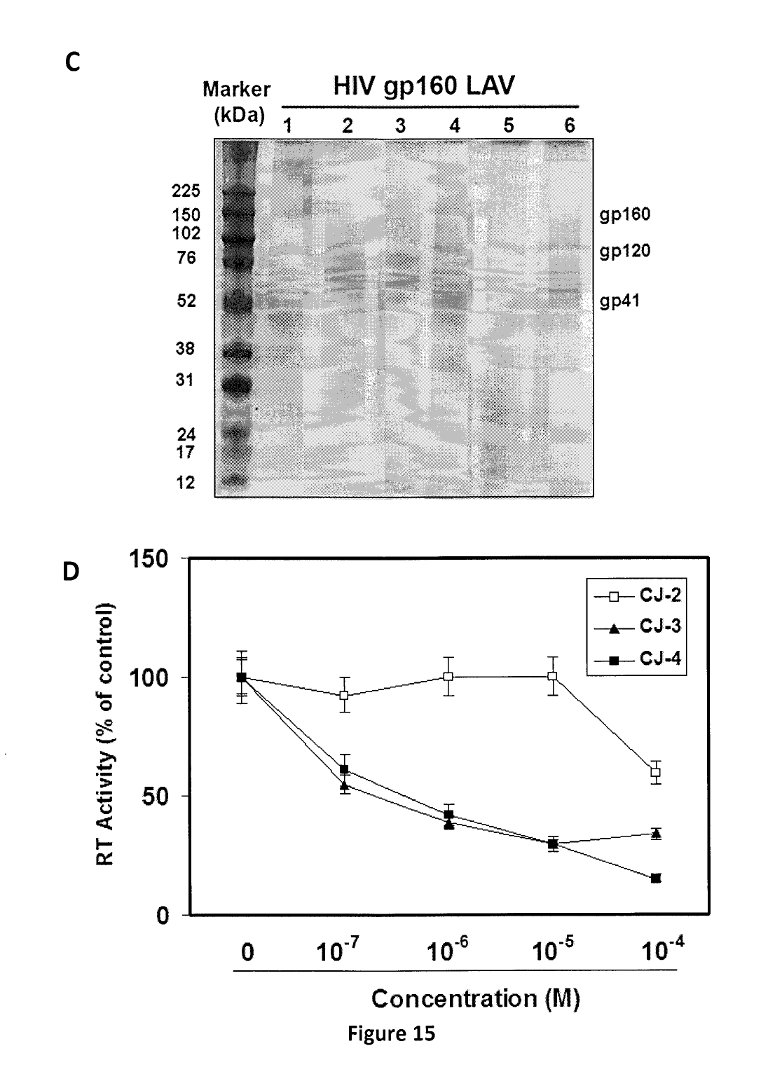

Cysteine residues in reduced stage are present in HIV envelop protein gp160. gp160 LAV was preincubated without (lanes 1, 3, 5) or with NEM biotin (lanes 2, 4, 6) and subjected to SDS page gel electrophoresis. The resulted proteins were subjected to silver stain to visualize the sizes (FIG. 15A) or Western blot for biotin antibody to identify the reductive cysteine residues in the protein (FIG. 15B).

Peptides were designed to disrupt HIV gp120 formation. The two peptides synthesized were: CJ-3 (VPVWKDAETTLFCASDAK; SEQ ID NO: 2) and CJ-4 (LVWEDLRNLCLFSYRLLR; SEQ ID NO: 3). HIV gp120 LAV preincubated without (lane 1) or with DTT (lane 2), L-cysteine (lane 3), CJ-3 (lane 4) CJ-4 (lane 5) or CJ-3 and CJ-4 (lane 6) was denatured and subjected to SDS PAGE gel electrophoresis. The resulted proteins were visualized by Coomassie blue staining (FIG. 15C). CJ-3 and CJ-4, alone or in combination, disrupted the structure of HIV gp120 LAV.

The HIV X4 strain NL4-3 was preincubated without (control) or with peptide CJ-2 which was a peptide with two cysteines in the hydrophilic domain, peptide CJ-3 which was derived from gp120, or peptide CJ-4 which was derived from gp41 for 1 hour at 37° C. at the concentrations indicated. The Jurkat T cells (0.5×10⁶ cell/ml) were infected by NL4-3 (30 ng/ml) for 16 hours and washed. The supernatants at day 6 postinfection was collected and subjected to HIV reverse transcriptase (RT) assay. The data are shown represent the mean±SD of duplicate cultures (FIG. 15D). Significantly, the HIV derived peptides, but not the control peptide, inhibited HIV.

### Example 3

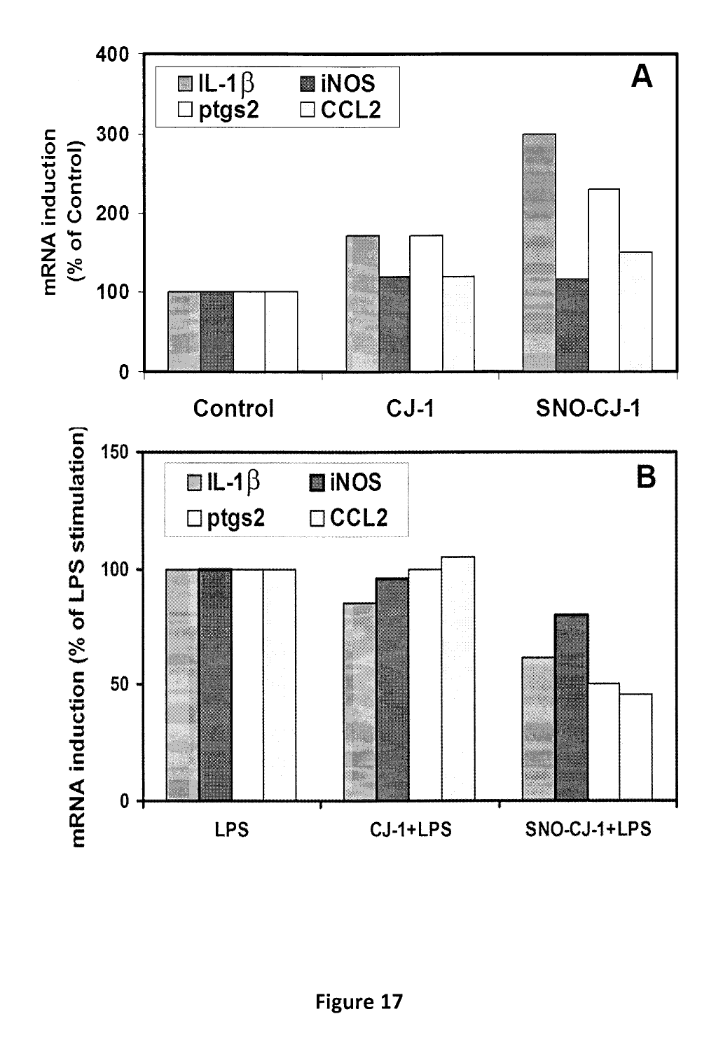

FIG. 17 shows that both CJ-1 and its nitrosylated form are capable of inducing transcription of inflammatory genes within macrophages. Notably, SNO-CJ-1 is more effective at inhibiting the effects of LPS-induced activation. More specifically, SNO-CJ-1 inhibits LPS induced mRNA expression of pro-inflammatory mediators in Raw264.7 macrophages. Raw 264.7 cells were incubated with or without CJ-1 (10 μM) or SNO-CJ-1 (10 μM) for 3 hours (FIG. 17A) or incubated with or without CJ-1 (10 μM) or SNO-CJ-1 (10 μM) for 20 minutes and treated with LPS (1 ng/mL) for an additional 3 hours (FIG. 17B). The cells were lysed and total RNA was isolated and subjected to real-time qPCR for mRNA expression of IL-1β, iNOS, ptgs2 and CCL2. SNO-CJ-1 inhibits mRNA expression of pro-inflammatory mediators.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated peptide comprising an amino acid sequence having at least 90% homology with a region of a target protein, wherein said region comprises a hydrophobic region which comprises at least one cysteine, and wherein said hydrophobic region comprises at least five hydrophobic amino acids.

2. The isolated peptide of claim 1, wherein said peptide is about 10 to about 30 amino acids in length.

3. The isolated peptide of claim 1, wherein said peptide is nitrosylated.

4. The isolated peptide of claim 1, wherein at least one cysteine residue within said hydrophobic region is nitrosylated.

5. The isolated peptide of claim 1, comprising a sequence having at least 90% homology with an amino acid sequence selected from the group consisting of SEQ ID NO: 1, SEQ ID NO: 2, and SEQ ID NO: 3.

6. A composition comprising at least one peptide of claim 1 and at least one pharmaceutically acceptable carrier.

7. The composition of claim 6, further comprising at least one anti-HIV compound.

8. A method for inhibiting an HIV infection in a subject in need thereof, said method comprising administering to said subject the composition of claim 6.

9. The method of claim 8, wherein the peptide comprises a sequence having at least 90% homology with SEQ ID NO: 2 or SEQ ID NO: 3.

10. The method of claim 8, further comprising the administration of at least one additional anti-HIV compound.

11. A method for inhibiting a pulmonary disease or disorder in a subject in need thereof, said method comprising administering to said subject the composition of claim 6.

12. The method of claim 11, wherein the peptide comprises a sequence having at least 90% homology with SEQ ID NO: 1.

13. A method of synthesizing a modulator of a multimeric protein, said method comprising
a) identifying a hydrophobic region within said multimeric protein, wherein said hydrophobic region comprises at least one cysteine, and
b) synthesizing a peptide comprising said hydrophobic region, wherein said peptide has at least 90% homology with said multimeric protein,
wherein the resultant peptide is said modulator of the multimeric protein.

14. A method of inhibiting multimeric protein assembly, said method comprising contacting the proteins of the multimeric complex with at least one peptide of claim 1, wherein the peptide comprises an amino acid sequence having at least 90% homology with a region of a target protein of the multimeric complex.

15. The method of claim 14, wherein said peptide is about 10 to about 30 amino acids in length.

16. The method of claim 14, wherein said peptide is nitrosylated.

17. The method of claim 16, wherein at least one cysteine residue of the hydrophobic region is nitrosylated.
